(19) United States
(12) Patent Application Publication
(10) Pub. No.: US 2026/0251544 A1
(43) Pub. Date: Aug. 27, 2026

(54) METHOD FOR CALCULATING SOLID-LIQUID TWO-PHASE VISCOSITY OF SLAG SYSTEM

(71) Applicant: Northeastern University, Shenyang City (CN)

(72) Inventors: Xin JIANG, Shenyang City (CN); Qingyu WANG, Shenyang City (CN); Yulu ZHOU, Shenyang City (CN); Fengman SHEN, Shenyang City (CN); Haiyan ZHENG, Shenyang City (CN); Qiangjian GAO, Shenyang City (CN)

(21) Appl. No.: 19/392,623

(22) Filed: Nov. 18, 2025

(30) Foreign Application Priority Data
Feb. 25, 2025 (CN) 202510208671.5

(51) Int. Cl. G01N 11/00

(52) U.S. Cl. CPC G01N 11/00; G01N 2011/0093

(57) ABSTRACT

A method for calculating solid-liquid two-phase viscosity of a slag system is provided, which belongs to the field of metallurgical engineering. The method for calculating the solid-liquid two-phase viscosity of the slag system includes the following steps: 1) determining a solid-phase content, a liquid-phase content, a solid-phase composition, and a liquid-phase composition of the slag system through an isothermal cross-section diagram of the slag system; 2) selecting an optimal liquid-phase viscosity model according to the liquid-phase composition to calculate the pure liquid-phase viscosity; and 3) selecting an optimal solid-liquid two-phase viscosity model according to the solid-phase content to calculate the solid-liquid two-phase viscosity.

### CROSS-REFERENCE TO RELATED PRESENT DISCLOSURE

This patent application claims the benefit and priority of Chinese Patent Application No. 202510208671.5 filed with the China National Intellectual Property Administration on Feb. 25, 2025, the disclosure of which is incorporated by reference herein in its entirety as part of the application.

### TECHNICAL FIELD

The present disclosure belongs to the field of metallurgical engineering, and in particular, relates to a method for calculating solid-liquid two-phase viscosity of a slag system.

### BACKGROUND

In the field of metallurgical engineering, slag viscosity is not only related to a smooth progress of a smelting process, but also affects transmission of heat and mass in the smelting process. Appropriate slag viscosity is a physical index that metallurgical workers are concerned about. The most direct method for obtaining slag viscosity is to perform measurement by experiments, but the viscosity of the slag system with coexisting solid and liquid phases is too high to be measured by a conventional method such as a capillary method, a falling ball method, a rotating cylinder method, an oscillation method, and a non-contact measurement method that utilizes a device such as an ultrasonic device, an electrostatic suspended device, and an air film suspended device.

### SUMMARY

The present disclosure provides a method for calculating solid-liquid two-phase viscosity of a slag system, which effectively improves the accuracy of calculation of solid-liquid two-phase viscosity, and helps optimize a metallurgical process, strengthen smelting, improve production efficiency and reduce energy consumption and CO₂ emission.

The technical solution of the present disclosure is as follows.

A method for calculating solid-liquid two-phase viscosity of a slag system includes the following steps:
step 1) determining a solid-phase content, a liquid-phase content, a solid-phase composition, and a liquid-phase composition of the slag system through an isothermal cross-section diagram of the slag system;
step 2) selecting an optimal liquid-phase viscosity model according to the liquid-phase composition to calculate the pure liquid-phase viscosity; and
step 3) selecting an optimal solid-liquid two-phase viscosity model according to the solid-phase content to calculate the solid-liquid two-phase viscosity.

In an embodiment, according to the method for calculating the solid-liquid two-phase viscosity of the slag system, step 1) is as follows:
when the slag system is located in a pure liquid-phase region, the liquid-phase composition is determined directly;
when the slag system is located in a two-phase region, the two-phase region includes a liquid phase and one solid phase, an only contact point between the two-phase region and the solid phase is a solid-phase contact point, and a straight line is determined by using a slag system component point and the solid-phase contact point, which intersects with the liquid-phase region to form a liquid-phase intersection point; a liquid-phase component at the liquid-phase intersection point is the liquid-phase content at the slag system component point, a solid-phase component at the solid-phase contact point is the solid-phase content at the slag system component point, and a ratio of a distance between the slag system component point and the liquid-phase intersection point to a distance between the slag system component point and the solid-phase contact point is a ratio of the solid-phase content to the liquid-phase content; and
when the slag system is located in a three-phase region, the three-phase region includes a liquid phase and two solid phases, an only contact point between the three-phase region and the liquid-phase region is a liquid-phase contact point, and a straight line is determined by using the slag system component point and the liquid-phase contact point, which intersects with the solid-phase region to form a solid-phase intersection point; a liquid-phase component at the liquid-phase contact point is the liquid-phase content at the slag system component point, a solid-phase component at the solid-phase intersection point is the solid-phase content at the slag system component point, and a ratio of a distance between the slag system component point and the liquid-phase contact point to a distance between the slag system component point and the solid-phase intersection point is a ratio of the solid-phase content to the liquid-phase content.

In an embodiment, according to the method for calculating the solid-liquid two-phase viscosity of the slag system, step 2) is as follows:
Riboud model is selected when the slag system is a multi-component complex system containing K₂O and Na₂O;
Urbain model is selected when the slag system is SiO₂—Al₂O₃—CaO—MgO four-component slag system and a sub-system thereof,
Kondratiev model is selected when the slag system is Al₂O₃—CaO—FeO—SiO₂ four-component slag system containing FeO and a sub-system thereof; and
in order to prevent deviation between a calculated viscosity value and a real viscosity value caused by an excessive number of components, it is necessary to correct a model calculation result, which means, a calculation result×a correction coefficient; where the correction coefficient ranges from 1.00 to 1.05 when the four-component slag system is calculated, the correction coefficient ranges from 1.05 to 1.10 when a five-component slag system is calculated, and the correction coefficient ranges from 1.10 to 1.15 when a slag system with six or more components is calculated.

In an embodiment, according to the method for calculating the solid-liquid two-phase viscosity of the slag system, step 3) is as follows:
Einstein model is selected when the solid-phase content ranges from 0 to 0.05;
Batchelor model is selected when the solid-phase content ranges from 0.05 to 0.5;
Roscoe model is selected when the solid-phase content ranges from 0.5 to 0.7;
Monney model is selected when the solid-phase content ranges from 0.7 to 1; and
in order to prevent deviation between the calculated viscosity value and the real viscosity value caused by an excessive solid-phase content generated, it is necessary to correct the model calculation result, which means, the calculation result×the correction coefficient; where the correction coefficient ranges from 0.95 to 1.00 when the Roscoe model is used, and the correction coefficient ranges from 0.90 to 0.95 when the Monney model is used.

The present disclosure has the following beneficial effects. According to the present disclosure, the isothermal cross-section diagram, the pure liquid-phase viscosity model and the solid-liquid two-phase viscosity model are jointly used, and different models and correction coefficients are selected in combination with specific slag systems, so that the problem that the viscosity of the solid-liquid two-phase slag system is too high to be directly measured by a testing device is solved, thereby effectively improving the accuracy of calculation of solid-liquid two-phase viscosity, and helping optimize a metallurgical process, strengthen smelting, improve production efficiency and reduce energy consumption and CO₂ emission.

### BRIEF DESCRIPTION OF THE DRAWINGS

FIG. 1 is an isothermal cross-section diagram of FeO—CaO—SiO₂ three-component slag system at 1320° C.

### DETAILED DESCRIPTION OF THE EMBODIMENTS

A method for calculating solid-liquid two-phase viscosity of a slag system includes the following steps 1) to 3).

In step 1), a solid-phase content, a liquid-phase content, a solid-phase composition, and a liquid-phase composition of the slag system are determined through an isothermal cross-section diagram of the slag system.

When the slag system is located in a pure liquid-phase region, the liquid-phase composition is determined directly. When the slag system is located in a two-phase region, the two-phase region includes a liquid phase and one solid phase, the only contact point between the two-phase region and the solid phase is a solid-phase contact point, and a straight line is determined by using a slag system component point and the solid-phase contact point, which intersects with the liquid-phase region to form a liquid-phase intersection point. A liquid-phase component at the liquid-phase intersection point is the liquid-phase content at the slag system component point, a solid-phase component at the solid-phase contact point is the solid-phase content at the slag system component point, and a ratio of a distance between the slag system component point and the liquid-phase intersection point to a distance between the slag system component point and the solid-phase contact point is a ratio of the solid-phase content to the liquid-phase content.

When the slag system is located in a three-phase region, the three-phase region includes a liquid phase and two solid phases, the only contact point between the three-phase region and the liquid-phase region is a liquid-phase contact point, and a straight line is determined by using the slag system component point and the liquid-phase contact point to intersect with the solid-phase region to form a solid-phase intersection point; a liquid-phase component at the liquid-phase contact point is the liquid-phase content at the slag system component point, a solid-phase component at the solid-phase intersection point is the solid-phase content at the slag system component point, and a ratio of a distance between the slag system component point and the liquid-phase contact point to a distance between the slag system component point and the solid-phase intersection point is a ratio of the solid-phase content to the liquid-phase content.

In step 2), an optimal liquid-phase viscosity model is selected according to the liquid-phase composition to calculate the pure liquid-phase viscosity.

Riboud model is selected when the slag system is a multi-component complex system containing K₂O and Na₂O.

The Riboud viscosity model is shown in Equation 1,

$${η}_{L} = A T {e}^{\frac{B}{T}} / 10 , \qquad ( 1 )$$

where η_{L} denotes the viscosity of the liquid slag in unit of Pa s; A denotes a pre-exponential factor; T denotes an absolute temperature in unit of K; and B denotes viscous flow activation energy in unit of j/mol.

The Riboud viscosity model divides the oxides in the melt into five types, and calculates the values of A and B by using the mole fraction of each oxide. The calculation method is shown in Equation 2 and Equation 3,

$$A = {e}^{- 17.51 + 1.73 {X}_{C a o} + 5.82 {X}_{C a {F}_{2}} + 7.02 {X}_{N {a}_{2} O} - 33.76 {X}_{A {l}_{2} {O}_{3}}} , \qquad ( 2 )$$

$$B = 31 \; 140 - 23896 {X}_{C a O} - 46356 {X}_{C a {F}_{2}} - 39159 {X}_{N {a}_{2} O} + 68833 {X}_{A {l}_{2} {O}_{3}} . \qquad ( 3 )$$

The calculation method of the mole fraction of five types of oxides is as follows:

$$1 ) {X}_{S i {O}_{2}} = {X}_{S i {O}_{2}} + {X}_{P {O}_{2.5}} + {X}_{T i {O}_{2}} + {X}_{Z r {O}_{2}} ;$$

$$2 ) {X}_{C a O} = {X}_{C a O} + {X}_{M g O} + {X}_{F e {O}_{1.5}} + {X}_{M n O} + {X}_{B {O}_{1.5}} ;$$

$$3 ) {X}_{C a {F}_{2}} = {X}_{C a {F}_{2}} ;$$

$$4 ) {X}_{N {a}_{2} O} = {X}_{N {a}_{2} O} + {X}_{{K}_{2} O} ;$$

$$5 ) {X}_{A {l}_{2} {O}_{3}} = {X}_{A {l}_{2} {O}_{3}} .$$

Urbain model is selected when the slag system is SiO₂—Al₂O₃—CaO—MgO four-component slag system and a sub-system thereof.

The Urbain viscosity model is shown in Equation 4,

$${η}_{L} = A T {e}^{\frac{1000 B}{T}} / 10. \qquad ( 4 )$$

The calculation method of the pre-exponential factor A is shown in Equation 5,

$$- ln A = mB / 1000 + n \qquad ( 5 )$$

where m and n are parameters, m=0.29; n=11.57.

The calculation method of the viscous flow activation energy B is shown in Equation 6,

$$B = \frac{{X}_{C} {B}_{C} + {X}_{M} {B}_{M}}{{X}_{C} + {X}_{M}} , \qquad ( 6 )$$

where X_{C} and X_{M} are the mole fractions of CaO and MgO, respectively; B_{C} and B_{M} are the viscous flow activation energies of CaO and MgO, respectively, in unit of J/mol.

The calculation methods of B_{C} and B_{M} are shown in Equation 7, Equation 8 and Equation 9,

$${B}_{C / M} = {B}_{0} + {B}_{1} {X}_{S} + {B}_{2} {X}_{S}^{2} + {B}_{3} {X}_{S}^{3} , \qquad ( 7 )$$

$${B}_{i = 0 , 1 , 2 , 3} = {a}_{i ( C / M )} + {b}_{i ( C / M )} α + {c}_{i ( C / M )} α , \qquad ( 8 )$$

$$α = \frac{{X}_{C} + {X}_{M}}{{X}_{C} + {X}_{M} + {X}_{A}} , \qquad ( 9 )$$

where X_{S} and X_{A} are mole fractions of SiO₂ and Al₂O₃; aᵢ, bᵢ and cᵢ are calculation parameters, and their values are shown in Table 1:

TABLE 1

| | aᵢ | | bᵢ | | cᵢ | |
|---|---|---|---|---|---|---|
| i | CaO | MgO | CaO | MgO | CaO | MgO |
| 0 | 13.20 | 13.20 | 41.50 | 15.90 | −45.00 | −18.60 |
| 1 | 30.50 | 30.50 | −117.20 | −54.10 | 130.00 | 33.00 |
| 2 | −40.40 | −40.40 | 232.10 | 138.00 | −298.60 | −112.00 |
| 3 | 60.80 | 60.80 | −156.40 | −99.80 | 213.60 | 97.60 |

Kondratiev model is selected when the slag system is Al₂O₃—CaO—FeO—SiO₂ four-component slag system containing FeO and a sub-system thereof.

The Kondratiev slag viscosity model is shown in Equation 10,

$${η}_{L} = A T {e}^{\frac{1000 B}{T}} . \qquad ( 10 )$$

The calculation method of the pre-exponential factor A is shown in Equation 11,

$$- ln A = m B + n , \qquad ( 11 )$$

where m and n are parameters; the value of parameter n is 9.322, and the calculation method of parameter m is shown in Equation 12,

$$m = {m}_{A} {X}_{A} + {m}_{C} {X}_{C} + {m}_{F} {X}_{F} + {m}_{S} {X}_{S} , \qquad ( 12 )$$

where m_{A}, m_{C}, m_{F} and m_{S} denote correction values of parameter m by Al₂O₃, CaO, FeO and SiO₂, and their values are shown in Table 2; and X_{A}, X_{C}, X_{F} and X_{S} are the mole fractions of Al₂O₃, CaO, FeO and SiO₂.

TABLE 2

| | | | | | | Correction | |
|---|---|---|---|---|---|---|---|
| | | i | | | | value of | |
| | j | 0 | 1 | 2 | 3 | parameter m | |
| bᵢ⁰ | 0 | 13.31 | 36.98 | −177.70 | 190.03 | n | 9.322 |
| bᵢ^{C}^{^{j}} | 1 | 5.50 | 96.20 | 117.94 | −219.56 | m_{A} | 0.370 |
| | 2 | −4.68 | −81.60 | −109.80 | 196.00 | m_{C} | 0.587 |
| bᵢ^{F}^{^{j}} | 1 | 34.30 | −143.64 | 368.94 | −254.85 | m_{F} | 0.665 |
| | 2 | −45.63 | 129.96 | −210.28 | 121.20 | m_{S} | 0.212 |

The calculation method of the viscous flow activation energy B is shown in Equation 13:

$$B = {\sum }_{i = 0}^{3} {b}_{i}^{0} {X}_{S}^{i} + {\sum }_{i = 0}^{3} {\sum }_{j = 1}^{2} ( {b}_{i}^{{C}^{j}} \frac{{X}_{C}}{{X}_{C} + {X}_{F}} + {b}_{i}^{{F}^{j}} \frac{{X}_{F}}{{X}_{C} + {X}_{F}} ) {α}^{j} {X}_{S}^{i} \qquad ( 13 )$$

where

$${b}_{i}^{0}$$

denotes the correction of parameter B by Al₂O₃—SiO₂ two-component slag system;

$${b}_{i}^{C}$$

and

$${b}_{i}^{F}$$

denote the correction of parameter B by CaO and FeO, and their values are shown in Table 2; and the calculation method of a is shown in Equation 14,

$$α = \frac{{X}_{C} + {X}_{F}}{{X}_{C} + {X}_{F} + {X}_{A}} . \qquad ( 14 )$$

In order to prevent deviation between a calculated viscosity value and a real viscosity value caused by an excessive number of components, it is necessary to correct a model calculation result, that is, a calculation result×a correction coefficient; where the correction coefficient ranges from 1.00 to 1.05 when the four-component slag system is calculated, the correction coefficient ranges from 1.05 to 1.10 when a five-component slag system is calculated, and the correction coefficient ranges from 1.10 to 1.15 when a slag system with six or more components is calculated.

In step 3), an optimal solid-liquid two-phase viscosity model is selected according to the solid-phase content to calculate the solid-liquid two-phase viscosity.

Einstein model is selected when the solid-phase content ranges from 0 to 0.05.

Batchelor model is selected when the solid-phase content ranges from 0.05 to 0.5.

Roscoe model is selected when the solid-phase content ranges from 0.5 to 0.7.

Monney model is selected when the solid-phase content ranges from 0.7 to 1.

In order to prevent deviation between the calculated viscosity value and the real viscosity value caused by an excessive solid-phase content generated, it is necessary to correct the model calculation result, that is, a calculation result×a correction coefficient; where the correction coefficient ranges from 0.95 to 1.00 when the Roscoe model is used, and the correction coefficient ranges from 0.90 to 0.95 when the Monney model is used.

### Embodiment

Taking the viscosity of three component points of FeO—CaO—SiO₂ three-component slag system at 1320° C. as an example, the calculated three component points are shown in Table 3.

TABLE 3

| Component | | | | |
|---|---|---|---|---|
| points | CaO/% | SiO₂/% | FeO/% | Temperature/° C. |
| A | 38.6 | 21.4 | 40 | 1320 |
| B | 25.7 | 1.43 | 60 | 1320 |
| C | 12.9 | 7.1 | 80 | 1320 |

### 1) Determination of the Liquid-Phase Composition and the Solid-Phase Content

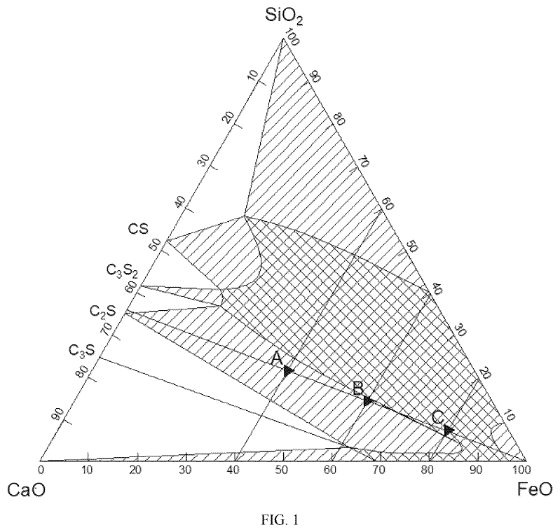

According to the slag system component and temperature, the isothermal cross-section diagram is drawn, and the drawing result is shown in FIG. 1. The liquid-phase component and the solid-phase content of the component points A, B and C read from FIG. 1 are shown in Table 4.

TABLE 4

| | | | | Liquid- | | | | Solid- |
|---|---|---|---|---|---|---|---|---|
| Compo- | Liquid-phase | | | phase | Solid-phase | | | phase |
| nent | composition/% | | | con- | composition/% | | | con- |
| points | CaO | SiO₂ | FeO | tent/% | CaO | SiO₂ | FeO | tent/% |
| A | 25 | 14 | 61 | 65 | 64 | 36 | 0 | 35 |
| B | 21 | 11 | 68 | 89 | 64 | 36 | 0 | 11 |
| C | 13 | 7 | 80 | 100 | 0 | 0 | 0 | 0 |

### 2) Model Selection and Viscosity Calculation

The Kondratiev model is selected because the slag system contains FeO. When the solid-liquid two-phase viscosity at this time is calculated, the Batchelor model should be selected when the solid-phase content is 35% and 11%, and the Einstein model should be selected when the solid-phase content is 0% (the viscosity when the solid-phase content is 0% is the liquid-phase viscosity, which is just an example here). The liquid-phase viscosity and the solid-liquid two-phase viscosity are calculated according to the corresponding models, respectively, and the calculation results are shown in Table 5 (no correction is needed at this time).

TABLE 5

| | | | Solid- | Calculation |
|---|---|---|---|---|
| | Liquid- | Calculation | liquid | result of |
| | phase | result of | two-phase | solid-liquid |
| Component | viscosity | liquid-phase | viscosity | two-phase |
| points | model | viscosity/Pa · s | model | viscosity/Pa · s |
| A | Kondratiev | 0.245 | Batchelor | 0.53 |
| B | Kondratiev | 0.196 | Batchelor | 0.23 |
| C | Kondratiev | 0.153 | Einstein | 0.15 |

That is the viscosity values of different component points of the slag system.

What is claimed is:

1. A method for calculating solid-liquid two-phase viscosity of a slag system, comprising:
step 1) determining a solid-phase content, a liquid-phase content, a solid-phase composition, and a liquid-phase composition of the slag system through an isothermal cross-section diagram of the slag system;
step 2) selecting a liquid-phase viscosity model according to the liquid-phase composition to calculate a pure liquid-phase viscosity; and
step 3) selecting a solid-liquid two-phase viscosity model according to the solid-phase content to calculate the solid-liquid two-phase viscosity.

2. The method for calculating the solid-liquid two-phase viscosity of the slag system according to claim 1, wherein step 1) comprises:
when the slag system is located in a pure liquid-phase region, determining the liquid-phase composition directly;
when the slag system is located in a two-phase region, in which a liquid phase and one solid phase are contained and an only contact point between the two-phase region and the solid phase is a solid-phase contact point, determining a straight line by using a slag system component point and the solid-phase contact point, which intersects with the liquid-phase region to form a liquid-phase intersection point, wherein a liquid-phase component at the liquid-phase intersection point is the liquid-phase content at the slag system component point, a solid-phase component at the solid-phase contact point is the solid-phase content at the slag system component point, and a ratio of a distance between the slag system component point and the liquid-phase intersection point to a distance between the slag system component point and the solid-phase contact point is a ratio of the solid-phase content to the liquid-phase content; and
when the slag system is located in a three-phase region, in which a liquid phase and two solid phases are contained and an only contact point between the three-phase region and the liquid-phase region is a liquid-phase contact point, determining a straight line by using the slag system component point and the liquid-phase contact point, which intersects with the solid-phase region to form a solid-phase intersection point, wherein a liquid-phase component at the liquid-phase contact point is the liquid-phase content at the slag system component point, a solid-phase component at the solid-phase intersection point is the solid-phase content at the slag system component point, and a ratio of a distance between the slag system component point and the liquid-phase contact point to a distance between the slag system component point and the solid-phase intersection point is a ratio of the solid-phase content to the liquid-phase content.

3. The method for calculating the solid-liquid two-phase viscosity of the slag system according to claim 1, wherein step 2) comprises:
selecting Riboud model when the slag system is a multi-component complex system containing K₂O and Na₂O;
selecting Urbain model when the slag system is SiO₂—Al₂O₃—CaO—MgO four-component slag system and a sub-system thereof,
selecting Kondratiev model when the slag system is Al₂O₃—CaO—FeO—SiO₂ four-component slag system containing FeO and a sub-system thereof, and
in order to prevent deviation between a calculated viscosity value and a real viscosity value caused by an excessive number of components, correcting a model calculation result through a calculation result×a correction coefficient; wherein the correction coefficient ranges from 1.00 to 1.05 when the four-component slag system is calculated, the correction coefficient ranges from 1.05 to 1.10 when a five-component slag system is calculated, and the correction coefficient ranges from 1.10 to 1.15 when a slag system with six or more components is calculated.

4. The method for calculating the solid-liquid two-phase viscosity of the slag system according to claim 1, wherein step 3) comprises:
selecting Einstein model when the solid-phase content ranges from 0 to 0.05;
selecting Batchelor model when the solid-phase content ranges from 0.05 to 0.5;
selecting Roscoe model when the solid-phase content ranges from 0.5 to 0.7;
selecting Monney model when the solid-phase content ranges from 0.7 to 1; and
in order to prevent deviation between the calculated viscosity value and the real viscosity value caused by an excessive solid-phase content generated, correcting the model calculation result through a calculation result×a correction coefficient; wherein the correction coefficient ranges from 0.95 to 1.00 when the Roscoe model is used, and the correction coefficient ranges from 0.90 to 0.95 when the Monney model is used.
